(19) United States
(12) Patent Application Publication
(10) Pub. No.: US 2013/0267882 A1
(43) Pub. Date: Oct. 10, 2013

(54) AMPUTATION DRESSINGS

(71) Applicant: Monica Ann Volker, Lake Geneva, WI (US)

(72) Inventor: Monica Ann Volker, Lake Geneva, WI (US)

(21) Appl. No.: 13/877,017

(22) PCT Filed: Sep. 29, 2011
(86) PCT No.: PCT/US2011/054006

§ 371 Date: Jun. 26, 2013

Related U.S. Application Data
(60) Provisional application No. 61/387,612, filed on Sep. 29, 2010.

(51) Int. Cl. A61F 15/00

(52) U.S. Cl. CPC A61F 15/006
USPC 602/79

(57) ABSTRACT

The invention relates to devices, systems and methods for caring for amputations (e.g., limb amputations, digit amputations). Specifically, the invention relates to devices and systems (e.g., dressing systems) for secure placement of wound dressing material (e.g., dressing material, bandage material) on the body of a subject.

The present application claims priority to U.S. Provisional Patent Application 61/387,612, filed Sep. 29, 2010, the disclosure of which is herein incorporated by reference in its entirety.

### FIELD OF THE INVENTION

The invention relates to devices, systems and methods for caring for amputations (e.g., limb amputations, digit amputations). Specifically, the invention relates to devices (e.g., dressing systems) for secure placement of wound dressing material (e.g., bandage material) on the body of a subject.

### BACKGROUND OF THE INVENTION

Amputation (e.g., of a limb, of a digit) is one of the oldest surgical procedures known to humankind, but carries tremendous immediate and long-term risk (e.g., of infection, chronic pain, abnormal or ineffective wound healing, psychological consequences, physical consequences secondary to loss of mobility). However, especially in situations where limb disease is so advanced as to preclude effective salvage or restoration attempts, amputation can be a life-saving technique and may be the treatment of choice for severe trauma, vascular disease, and tumors.

The incidence of amputation in the United States is significant. Over 134,000 lower-limb amputations are performed each year (Smith et al. (2003) J. Rehabil. Research and Dev. 40:213-224; herein incorporated by reference in its entirety). The incidence of amputation is projected to rise substantially in decades to come, due in large part to climbing rates of type 2 diabetes and related pathologies. A study based on data from 2005 calculated that one out of every 190 Americans was living with loss of a limb, and projected that this rate would double by the year 2050 (Ziegler-Graham et al. (2008) Arch Phys Med Rehabil. 89(3):422-9; herein incorporated by reference in its entirety).

Post-surgical complications of amputation include, but are not limited to, wound breakdown and skin problems, swelling, edema, joint contractures, pain, and phantom limb sensation. The immediate post-surgical period presents considerable risk of infection, and despite the long history behind amputation surgical procedures there is little consensus on best practices for post-surgical management of amputation wounds and limb stumps. Challenges in post-surgical management of the wound site include, but are not limited to, preventing contracture of the proximal joint, reducing edema, facilitating primary and secondary wound healing, pain management, preventing infection. Both soft dressing and rigid dressings have been utilized for immediate post-surgical wound treatment (Smith et al. (2003) J. Rehabil. Research and Dev. 40:213-224; herein incorporated by reference in its entirety), with varying success reported for each. Soft dressings can be associated with uncomplicated healing outcomes at relatively low cost; however, management of edema can be difficult with soft dressings. One strategy to prevent limb stump edema is to affix the margins of soft dressings by encircling the limb stump with wrapping material such as elastic bandages. However, this may cause limb stump strangulation, thereby impairing skin survival and healing. Additionally, care providers face the difficulty of trying to maintain adequate wound access while controlling edema and preventing infection in face of the tendency of soft dressings to loosen and fall off.

Improved dressing systems for post-surgical limb amputations are needed.

### SUMMARY OF THE INVENTION

The invention relates to devices, systems and methods for caring for amputations (e.g., limb amputations, digit amputations). Specifically, the invention relates to devices (e.g., dressing systems) for secure placement of wound dressing material (e.g., bandage material) on the body of a subject.

Post-surgical management of limb amputation wounds and limb stumps necessitates frequent changes of wound dressing materials to prevent infection and to promote wound healing. Affixing soft dressing materials to the limb stump without creating localized points of unacceptably low or high pressure, however, can be difficult. For example, wrapping soft dressing material with elastic bandage material circumferentially around the distal portion of an amputation limb stump such that the dressing material is held firmly in place can result in impaired circulation and limb strangulation, thereby hindering healing. However, failure to secure bandage material in place adequately can result in loosening and loss of the bandage, increasing the risk of wound infection, edema, and dehiscence. The present invention provides dressing systems for post-surgical amputation wounds that secure the dressing materials in place while permitting ready access to the wound.

In preferred embodiments, one or more stabilizing belts circumscribe an anatomical location other than the amputated (e.g., limb, digit) stump and are configured to anchor the dressing system securely. In preferred embodiments, dressing materials contacting the wound site are removably connected with the one or more stabilizing belts. In some preferred embodiments, connectors (e.g., straps, bands, belts, ribbons, fasteners, ties, etc.) span the dressing materials and the one or more stabilizing belts. In particularly preferred embodiments, the dressing materials may be removed from the connectors. In some embodiments, the dressing system comprises fasteners (e.g., hooks, eyes, buttons, hook-and-loop fasteners (e.g., Velcro® fasteners), adhesive, pins, tabs, snaps, clasps, clamps, carabiners, self-gripping devices, zippers, prongs, eyelets, poppers, ties, and the like) within one element of the system or between two elements of the system. In some preferred embodiments, the dressing materials are contacted by backing material on the non-wound-facing side of the dressing material. In preferred embodiments, the backing material imparts form to the dressing material, thereby improving the fit or functionality of the dressing material. Additional aspects of some dressing system elements are further described herein.

The invention is not limited by the dimensions of the one or more stabilizing belts. Stabilizing belts may be less than 0.5 cm, 0.5-1 cm, 1-2 cm, 2-3 cm, 3-4 cm, 4-5 cm, 5-10 cm, 10-15 cm, 15 or more cm in width. The invention is not limited by the anatomical location that the stabilizing belt is configured to encompass. The stabilizing belt may encompass a neck, chest, torso, shoulder, waist, hips, head, or any other non-limb stump location or a combination thereof. In some embodiments, more than one stabilizing belt is used. In some embodiments, the more than one stabilizing belt may intersect with each other at an angle, without limitation to the angle of intersection. The angle of intersection may be less than 10 degrees, 10-20 degrees, 20-30 degrees, 30-40 degrees, 40-50 degrees, 50-60 degrees, 60-70 degrees, 70-80 degrees, 80-90 degrees. In some embodiments, the stabilizing belt is adjustable in length. In some embodiments, the stabilizing belt is not adjustable in length.

The invention is not limited by the form of the connectors spanning the one or more stabilizing belts and the soft dressing materials. The connectors may be 1, 2, 3, 4, 5, 6, 7, 8, 9, or more in number. The invention is not limited by the dimensions of the connectors. The connectors may be less than 0.5 cm, 0.5-1 cm, 1-2 cm, 2-3 cm, 3-4 cm, 4-5 cm, 5-10 cm, 10-15 cm, 15 or more cm in width. In some preferred embodiments, the connectors are elongated (e.g., straps, strips, ribbons, cords, ties, etc.). In some embodiments, the connectors are adjustable in length. In some embodiments, the connectors are not adjustable in length. Where more than one connector is used, their dimensions (e.g., length) may match or differ. In preferred embodiments, the dimensions (e.g., length) of the connector(s) are configured to hold the soft dressing material in close contact with the wound site. In some preferred embodiments, the dimensions (e.g., length) of the connector(s) may be configured to provide sufficient pressure to the wound site (e.g., amputation site, limb stump, digit stump) to prevent edema. In some preferred embodiments, the connectors are configured to prevent localized points of undesirably high pressure (e.g., uneven pressure leading to impaired circulation or limb stump strangulation).

In some embodiments, the dressing system comprises fasteners (e.g., hooks, eyes, buttons, hook-and-loop fasteners (e.g., Velcro® fasteners), adhesive, pins, tabs, snaps, clasps, clamps, carabiners, self-gripping devices, zippers, prongs, eyelets, poppers, ties, and the like) within one element of the system (e.g., one or more fastener may be located on the dressing material or backing material). In some embodiments, the dressing system comprises fasteners connecting two different elements of the system (e.g., one or more fastener may span dressing material and a connector, a connecter and a stabilizing belt, etc.) In some embodiments, fasteners within a single element or between two elements serve to adjust fit or function of the system (e.g., improves wound contacting pressure, improves contact between dressing material and wound, stump pressure, pressure directed to other parts of the body in direct or indirect contact with the dressing system). In one non-limiting example, dressing material and/or dressing material backing may comprise fasteners (e.g., tabs) that may be removably placed within the dressing system to customize fit, and/or adjust pressure (e.g., pressure at the wound surface, pressure at a location proximal to the wound surface, pressure at a location distal to the wound surface).

In preferred embodiments, dressing materials contact the wound (e.g., limb stump) site. The invention is not limited by dressing materials used. Dressing materials include, but are not limited to, gauze, cotton, cloth, non-cloth (e.g., non-woven) material, films, gels, foams, hydrocolloids, alginates, hydrogels and polysaccharide pastes, granules, beads. Dressing materials may be dry, moist, or wet. Where moist or wet dressing materials are used, they are not limited by their fluid (e.g., water, saline) content. In some embodiments, more than one type of dressing material is used. In particularly preferred embodiments, dressing materials are sterile. In some embodiments, dressing materials used to contact the wound are contacted by non-dressing materials (e.g., backing material). In some embodiments, the non-dressing (e.g., backing) material provides further shape or functionality (e.g., absorption, temperature regulation, permeability, positioning aid). In particularly preferred embodiments, the dressing material is configured to be disposable. In some embodiments, the dressing material and backing material are disposable as a unit. In some embodiments, the dressing material in contact with backing material is disposed, while the dressing material is retained for repeated use. In some embodiments, the dressing and/or non-dressing (e.g., backing) material is configured to accommodate therapeutic compositions or devices (e.g, ointment, salve, powder, tincture, temperature-regulating devices, neurostimulatory devices, etc.).

In some embodiments, the dressing system (e.g., dressing material, backing material, stabilizing belts, connectors, fasteners) includes graphical elements (e.g., two-dimensional markings (e.g., numbers, letters, decorations, symbols, lines, etc.), three-dimensional graphical features (e.g., stitching, etching, raised ornamentation, or the like). In some embodiments, the graphical elements serve as visual reference point(s), e.g., for placement of the system elements. In some embodiments, the graphical elements facilitate consistent re-placement of system elements, e.g., after old dressing material is removed and new dressing material is applied.

The present invention is not limited by materials used for construction of embodiments of the invention. In some embodiments, the device is manufactured out of cloth textile(s). Textiles used for construction may be made from natural materials (e.g., wool, silk, cotton, jute, linen, hemp, bamboo, flax), synthetic materials (e.g., polyester, acetate, acrylic, nylon, spandex, olefin fiber, polylactide fiber, milk fiber, casein fiber), or a mixture of natural and synthetic materials. Textiles are not limited by the nature of thread count, warp, weave, weight, or other characteristics. In some embodiments, materials used for devices and systems of the present invention are capable of unidirectional extension. In some embodiments, Veltex® brand fabric is used. In some embodiments, non-woven polymers or composites are used for construction (e.g., rubber, silicone, neoprene). In some embodiments, more than one material is used for construction.

In some embodiments, the stabilizing belt is configured to encompass a trunk (e.g., waist, hips, chest) of a subject, e.g., a subject with a lower limb amputation. In some embodiments, a stabilizing belt is configured to encompass a trunk (e.g., pectoral region, chest) without regard to the specific angle at which the trunk is configured. In some embodiments, a stabilizing belt passes over a shoulder, across a trunk (e.g., chest), and emerges underneath a limb (e.g., an arm, a limb stump) to form a loop diagonally across the chest of a subject (e.g., a subject with an upper limb amputation).

It is an aim of the present invention to provide a post-surgical dressing system for a subject that has experienced an amputation (e.g., limb amputation, digit amputation) wherein the system reduces the risk of limb or digit stump strangulation, edema, infection, and/or tourniquet effect. It is a further aim to provide a system that facilitates wound access by healthcare personnel and aids in inspection of post-surgical wound, e.g., for monitoring infection, healing process, joint contracture, etc. It is a further aim to reduce the time and cost involved in providing post-surgical care to amputation patients.

Devices and systems of the present invention are not limited by the type of subject on which they are used. The subject may be an animal (e.g., livestock, companion animal) or a human. In some preferred embodiments, the subject is a human. Devices and systems of the present invention are not limited by the age of the subject on which they are used. The subject may be pediatric, adult, of advanced age (e.g., geriatric). Devices and systems of the present invention may be configured for subjects of a variety of bodily dimensions (e.g., pediatric subjects, subjects of small stature, subjects of tall stature, obese subjects, subjects with congenital defects, etc.)

The present invention is not limited by the type of amputation wound to which it is applied. Types of amputations include, but are not limited to, leg amputations (e.g., digit (e.g., toe) amputations, partial foot amputation (Chopart, Lisfranc, Ray), ankle disarticulation (Syme, Pyrogoff), below-knee (BK) amputation (transtibial amputation (TTA), e.g. Burgess, Kingsley Robinson), knee-bearing amputation (knee disarticulation, e.g. Gritti or Gritti-Stokes), above knee (AK) amputation (transfemoral), Van-ness rotation/rotationplasty (wherein a foot is rotated and reattached to allow the ankle joint to be used as a knee), hip disarticulation, hemipelvectomy/hindquarter amputation), arm amputations (e.g., digit (e.g., thumb, finger) amputation, metacarpal amputation, wrist disarticulation, forearm amputation (transradial), elbow disarticulation, above-elbow (AE) amputation (transhumeral), shoulder disarticulation, forequarter amputation, Krukenberg procedure), hemicorporectomy.

In certain embodiments, the present invention provides a dressing system for treatment of a subject with an amputation comprising a) a stabilizing belt configured to encompass a portion of said subject's body, wherein the portion of the subject's body is not an amputation stump, b) dressing material configured to contact an amputation site of the subject, and c) one or more connectors contacting and spanning the stabilizing belt and the dressing material. In some embodiments, the stabilizing belt is configured to encompass a location such as a torso, waist, hips, neck, and/or chest. In some embodiments, the amputation is a type such as an upper limb amputation, a lower limb amputation, or a digit amputation. In some embodiments, the system further comprises backing material in contact with the dressing material. In some embodiments, the one or more fasteners comprises a structure of a type such as hooks, eyes, buttons, hook-and-loop fasteners, adhesive, pins, tabs, snaps, clasps, clamps, carabiners, self-gripping devices, zippers, prongs, eyelets, poppers, or ties. In some embodiments, the one or more fasteners are configured to connect the one or more connectors with the dressing material. In some embodiments, the backing material further comprises at least one fastener of a type such as hooks, eyes, buttons, hook-and-loop fasteners, adhesive, pins, tabs, snaps, clasps, clamps, carabiners, self-gripping devices, zippers, prongs, eyelets, poppers, or ties. In some embodiments, the dressing system comprises graphical elements, wherein the graphical elements serve as visual reference points for adjustment of fit. In some embodiments, the stabilizing belt is configured to be adjustable in its length. In some embodiments, at least one of the one or more connectors is configured to be adjustable in its length.

Additional embodiments will be apparent to persons skilled in the relevant art based on the teachings contained herein.

### DESCRIPTION OF THE DRAWINGS

FIGS. 1A and 1B shows an embodiment configured for a subject with lower limb amputation. 1A, dressing system. 1B, detail of distal portion of the dressing system.

FIG. 2 shows an embodiment configured for a subject with upper limb amputation.

### DEFINITIONS

To facilitate an understanding of the present invention, a number of terms and phrases are defined below:

As used herein, the terms “subject” and “patient” refer to any animal, such as a mammal like a dog, cat, bird, livestock, and preferably a human (e.g. a human who has experienced an amputation).

As used herein, the term “stabilizing belt” refers to a loop structure that encompasses a non-limb-stump anatomical location on a subject, e.g., trunk, torso, chest, pectoral region, waist, hips, neck, shoulder, head, combination thereof. Stabilizing belts are not limited by the materials with which they are constructed, nor by their shape or form. In preferred embodiments, a stabilizing belt forms a continuous loop, although a continuous stabilizing belt may include structures that permit dimension (e.g., length) adjustment (e.g., buckles, clasps, hooks, buttons, tabs, pleats, and the like).

As used herein, the term “connector” refers to a structure that contacts a stabilizing belt and soft dressing material or backing that is in contact with soft dressing material. Connectors are not limited by the materials with which they are constructed, nor by their shape or form. Connectors may be elongated (e.g., straps, strips, ribbons, cords, ties, etc.). In some preferred embodiments, connectors comprise fasteners, e.g., hooks, eyes, buttons, hook-and-loop fasteners (e.g., Velcro® fasteners), adhesive, pins, tabs, snaps, clasps, clamps, carabiners, self-gripping devices, zippers, prongs, eyelets, poppers, ties, and the like for interaction with soft dressing material, soft dressing material backing, and/or a stabilizing belt.

As used herein, the term “dressing”, “soft dressing material” and “dressing material” refers to material contacting a wound site (e.g., amputation wound site) that is designed to cover, protect, and/or facilitate healing. In some cases, dressing material is absorbent. Dressing material includes, but is not limited to, gauze, cotton, cloth, non-cloth (e.g., non-woven) material, films, gels, foams, hydrocolloids, alginates, hydrogels and polysaccharide pastes, granules, beads.

As used herein, the term “fastener” refers to a structure that connects two elements of a dressing system, e.g., a structure that connects wound dressing material or wound dressing material backing and a connector; a structure that connects a stabilizing belt and a connector, or that connect two regions within a single element (e.g., two regions of dressing material, two regions of backing material) to adjust fit or function of the system (e.g., improves wound contacting pressure, improves contact between dressing material and wound, stump pressure, pressure directed to other parts of the body in direct or indirect contact with the dressing system). In some embodiments, fasteners facilitate removal of one or more element of a wound dressing system. Fasteners include, but are not limited to, hooks, eyes, buttons, hook-and-loop fasteners (e.g., Velcro® fasteners), adhesive, pins, tabs, snaps, clasps, clamps, carabiners, self-gripping devices, zippers, prongs, eyelets, poppers, ties, and the like.

As used herein, the term “backing”, “backing material”, “soft dressing backing”, “wound material backing” and similar terms refer to a material that contacts soft dressing material to impart form or structure. Backing material may be a textile or a non-textile material. In some embodiments, backing is partially or completely impermeable to fluid, e.g., serving to contain wound exudate, blood, or other bodily fluids.

### DETAILED DESCRIPTION OF THE INVENTION

The invention relates to devices, systems and methods for post-surgical treatment of subjects experiencing an amputation (e.g., limb amputation, digit amputation). Specifically, the invention relates to devices (e.g., dressing systems) for secure placement of wound dressing material (e.g., bandage material) on the body of a subject.

While the post-surgical period is a critical time for healing and a determination of long-term recovery, there is little consensus as to best practices for post-surgical care of amputation wound sites. Post-surgical management of amputation wounds necessitates frequent changes of dressing materials to prevent infection and to promote wound healing. Affixing soft dressing materials to the limb stump without creating localized points of high pressure, however, can be problematic. For example, wrapping soft dressing material with elastic bandage material circumferentially around the distal portion of an amputation limb stump to hold dressing material firmly can result in impaired circulation and limb strangulation, thereby hindering healing. However, failure to secure dressing material adequately can result in loosening, increasing the risk of wound infection and dehiscence. The present invention provides dressing systems for post-surgical amputation wounds that secure the dressing materials in place while permitting ready access to the wound. In preferred embodiments, one or more stabilizing belts circumscribe an anatomical location other than the amputated (e.g., limb, digit) stump and are configured to anchor the dressing system securely. In preferred embodiments, dressing materials contacting the wound site are removably connected with the one or more stabilizing belts. In some preferred embodiments, connectors (e.g., straps, bands, belts, ribbons, fasteners, ties, etc.) span the soft dressing materials and the one or more stabilizing belts. In particularly preferred embodiments, the soft dressing materials may be removed from the connectors (e.g., the soft dressing materials are removably fastened to one or more connectors using hooks, eyes, buttons, hook-and-loop fasteners (e.g., Velcro® fasteners), adhesive, pins, tabs, snaps, clasps, clamps, carabiners, self-gripping devices, zippers, prongs, eyelets, poppers, ties, and the like).

Amputations

Amputation is the surgical removal of a body extremity. As a surgical measure, it is used to control pain or a disease process in the affected limb, such as malignancy or gangrene. In some cases, it is carried out on individuals as a preventative measure.

Amputations can generally be categorized as upper extremity amputations (including upper extremity digit amputations) and lower extremity amputations (including lower extremity digit amputations). Lower-extremity amputations may be performed for conditions including, but not limited to, peripheral vascular disease (PVD), including that arising from diabetes mellitus; trauma; tumors; infections; congenital limb deficiency. Similarly, upper-extremity amputations may be performed for conditions including, but not limited to, vascular disease or conditions (e.g., vascular occlusion, vascular compromise, embolic occlusion, chronic ischemia, chronic vascular insufficiency), thermal burns, chemical burns, frostbite, gangrene, trauma, tumors, sepsis. These conditions are considered in more detail infra:

A common cause of lower extremity amputations in the United States is peripheral vascular disease (ischemic disease), primarily in elderly persons with diabetes mellitus. These patients often experience peripheral neuropathy that progresses to trophic ulcers and subsequent gangrene and osteomyelitis. Similarly, upper extremity amputations may be required when severe vascular deficiency is present.

An additional cause of upper and lower extremity amputations is trauma. For example, severe open (IIIc) fractures with popliteal artery and posterior tibial nerve injuries can be treated with current techniques; however, treatment is at a high cost, and multiple surgeries are required. The result is often a leg that is painful, nonfunctional, and less efficient than a prosthesis. Trauma precipitating upper and lower extremity amputations may be experienced in settings including, but not limited to, vehicle accidents; industrial accidents; military service; search-and-rescue operations; natural disasters; and events leading to crushing injuries (e.g., building collapse, mining injuries, and the like).

Tumors in lower or upper extremities may require surgical amputation of the affected limb as a life-sparing technique, although the advent of advanced limb-salvage techniques has reduced the incidence of tumor-precipitated amputations.

Treatment of sepsis with vasoconstrictor agents may at times lead to vessel occlusion and subsequent extremity necrosis, necessitating amputation. At other times, eradication of infection from many difficult sources necessitates removal of the affected digit or limbs.

Amputations for congenital limb deficiencies are performed primarily in the pediatric population because of failure of partial or complete formation of a portion of the limb. Congenital extremity deficiencies have been classified as longitudinal, transverse, or intercalary. Radial or tibial deficiencies are referred to as preaxial, and ulnar and fibular deficiencies are referred to as postaxial.

Dressing Materials

Wound dressing materials are available in a variety of forms, and the choice of dressing material depends on factors including, but not limited to, the amount of wound exudate present, the risk of infection, the age of the wound, the condition (e.g., immune status, mobility level) of the subject, and other confounding factors (e.g., the type of wound, skin disorders, burns).

A polyvinyl film dressing (e.g., OpSite (Smith & Nephew), Tegaderm (3M)), which is semipermeable to oxygen and moisture and impermeable to bacteria, may find use for wounds that are neither dry nor highly exudative. For dry wounds, hydrocolloid dressings, such as DuoDerm or IntraSite hydrocolloid, are impermeable to oxygen, moisture, and bacteria. Such materials maintain a moist environment and support autolytic debridement, and are appropriate for relatively desiccated wounds. For exudative wounds, absorptive dressings, such as calcium alginates (e.g., Kaltostat (Calgon Vestal), Curasorb (Kendall)) and hydrofiber dressings (e.g., Aquacel and Aquacel-AG (Convatec)), are highly absorptive and are appropriate for exudative wounds. Alginates are available in rope form, which is useful for packing deep wounds. For very exudative wounds, impregnated gauze dressings, such as Mesalt (Scott), are useful. Twice-daily dressing changes may be needed. For infected wounds, silver sulfadiazine (Silvadene) may be used if the subject is not allergic to sulfa drugs. If the subject is allergic to sulfa, bacitracin-zinc ointment or an ionic-silver hydrofiber dressing (e.g., Aquacel-AG) may be used.

In some cases, bandaging a challenging anatomic area requires a highly conformable dressing, such as an extra-thin hydrocolloid. Securing a dressing in a highly moist challenging site requires a conformable and highly adherent dressing, such as a wafer hydrocolloid. Hydrogel sheets and nonadhesive forms are useful for securing a wound dressing when the surrounding skin is fragile.

Wound dressing materials may include, but are not limited to, gauze, cotton, cloth, non-cloth (e.g., non-woven) material, films, gels, foams, hydrocolloids, alginates, hydrogels and polysaccharide pastes, granules, beads. Additional examples of wound dressing materials are given in Table 1.

TABLE 1
Wound dressing materials

| Category | Examples | Description | Applications |
|---|---|---|---|
| Alginate | AlgiSite, Comfeel, | Alginate dressings are made of seaweed | These dressing are highly |
| | Curasorb, | extract contains guluronic and mannuronic | absorbent and useful for |
| | Kaltogel, Kaltostat, | acids that provide tensile strength and | wounds have copious |
| | Sorbsan, Tegagel | calcium and sodium alginates, which | exudate. Alginate rope is |
| | | confer an absorptive capacity. Some can | particularly useful to pack |
| | | leave fibers in the wound if they are not | exudative wound cavities or |
| | | thoroughly irrigated. These dressings are | sinus tracts. |
| | | secured with secondary coverage. | |
| Hydrofiber | Aquacel, Aquacel- | An absorptive textile fiber pad, hydrofiber is | Hydrofiber absorbent |
| | Ag, Versiva | also available as a ribbon for packing of | dressings used for exudative |
| | | deep wounds. This material is covered with | wounds. |
| | | a secondary dressing. The hydrofiber | |
| | | combines with wound exudate to produce a | |
| | | hydrophilic gel. Aquacel-Ag contains 1.2% | |
| | | ionic silver that has strong antimicrobial | |
| | | properties against many organisms, | |
| | | including methicillin-resistant | |
| | | Staphylococcus aureus and vancomycin- | |
| | | resistant enterococci. | |
| Debriding | Hypergel | Varous products provide some chemical or | Debriding agents are useful |
| agents | (hypertonic saline | enzymatic debridement. | for necrotic wounds as an |
| | gel), Santyl | | adjuct to surgical |
| | (collagenase), | | debridement |
| | Accuzyme (papain | | |
| | urea) | | |
| Foam | LYOfoam, | Polyurethane foam has absorptive | These dressings are useful |
| | Spyrosorb, Abrevyn | capacity. | for cleaning granulating |
| | | | wounds with minimal |
| | | | exudate. |
| Hydrocolloid | CombiDERM, | Hydrocolloid dressings are made of | Hydrocolloid dressings are |
| | Comfeel, | microgranular suspension of natural or | useful for dry necrotic |
| | DuoDerm CGF | synthetic polymers, such as gelatin or | wounds, wounds with |
| | Extra Thin | pectin, in an adhesive matrix. The granules | minimal exudate and for |
| | Granuflex, | change from a semihydrated state to a gel | clean granulating wounds. |
| | Tegasorb | as the wound exudate is absorbed. | |
| Hydrogel | Aquasorb, | Hydrogel dressings are water-based or | These dressings are useful |
| | DuoDerm, Intrasite | glycerin-based semipermeable hydrophilic | for dry, sloughy, necrotic |
| | Gel, Granugel, | polymers; cooling properties may decrease | wounds (eschar) |
| | Normlgel, Nu-Gel, | wound pain. These gels can lose or absorb | |
| | Purilon Gel, KY | water depending upon the state of | |
| | Jelly | hydration of the wound. They are secured | |
| | | with secondary covering. | |
| Low- | Mepore, Skintact | Low-adherence dressings are made of | These dressings are useful |
| adherence | Release | various materials designed to remove | for acute minor wounds, such |
| dressing | | easily without damaging underlying skin. | as skin tears, or a final |
| | | | dressing for chronic wounds |
| | | | that have nearly healed. |
| Transparent | OpSite, Skintact | Transparent films are highly conformable | These dressings are useful |
| film | Release, | acrylic adhesive films with no absorptive | for clean, dry wounds with |
| | Tegaderm, | capacity and little hydrating ability. They | minimal exudate. They also |
| | Bioclusive | may be vapor permeable or perforated. | are used to secure an |
| | | | underlying absorptive |
| | | | material, to protect high- |
| | | | friction areas and areas that |
| | | | are difficult to bandage (eg, |
| | | | heels) and to secure |
| | | | intravenous catheters. |

Description of Embodiment for Use in Lower Limb Amputation

The following examples are provided in order to demonstrate and further illustrate a preferred embodiment and certain aspects of the present invention and is not to be construed as limiting the scope thereof.

In the following figures, the same reference numerals are used to identify the same components in the various views and alternate embodiments.

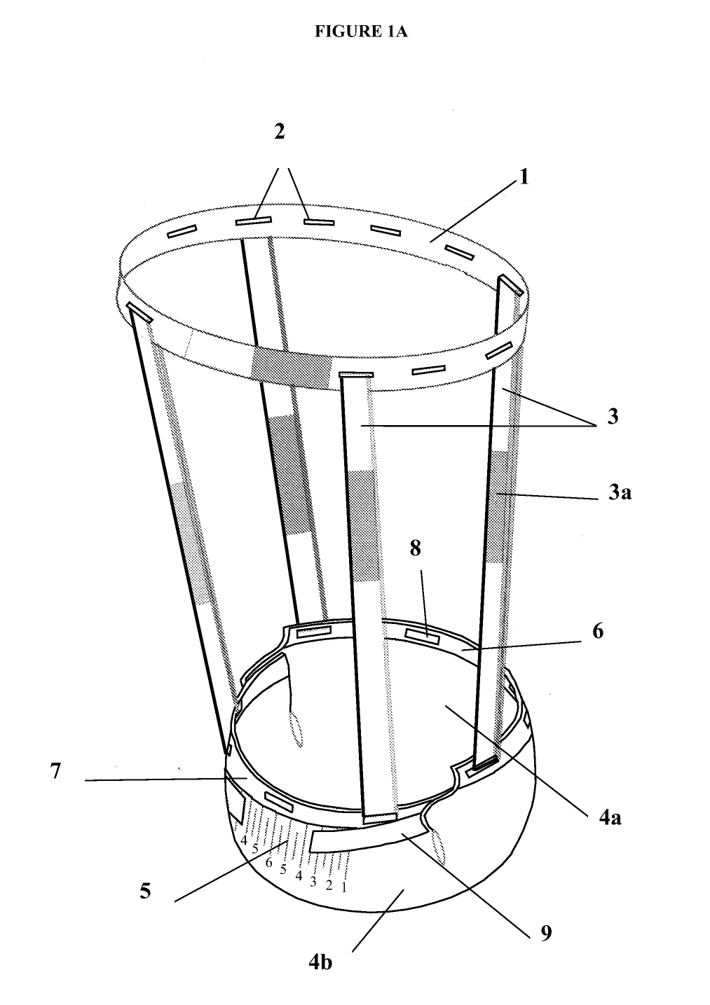

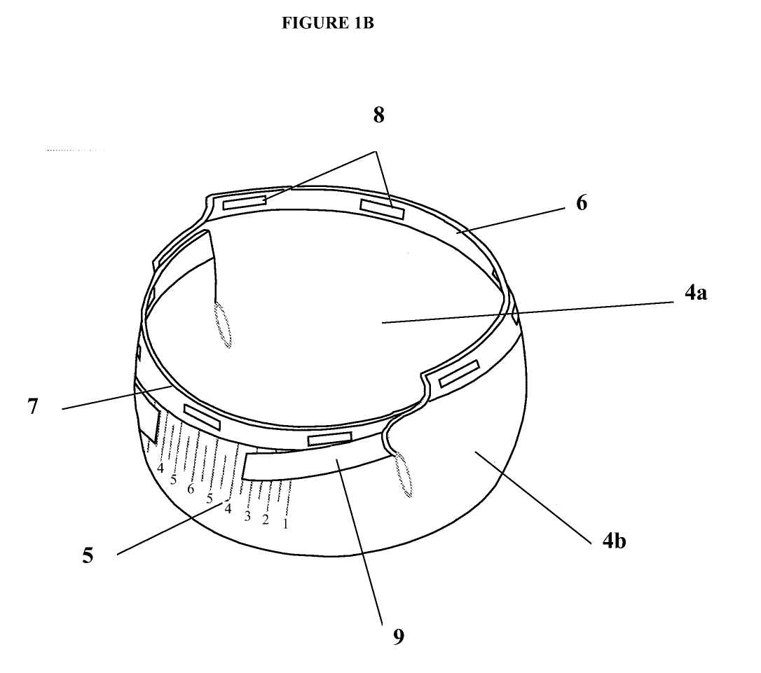

Referring now to FIG. 1, there is generally illustrated an article of manufacture (e.g., a wound dressing system) configured for use with a subject having a lower extremity amputation. The embodiment includes a stabilizing belt 1 comprising a plurality of slits 2 through which connectors are threaded 3. The connectors are in the form of loops that are fastened by hook-and-loop fasteners 3a, which confers the ability to adjust the length of each connector individually for customization of fit. A wound dressing material surface 4a is configured to contact the wound site of a lower extremity (e.g., above the knee) amputation wound on the interior face of the wound dressing material, and on the opposite face is contacted by backing material 4b. The exterior non-wound-facing surface of the backing material 4b is elaborated with graphical features 5. The posteriorly and anteriorly-oriented edges of the wound dressing material are bordered by reinforced edges 6, 7 that comprise a plurality of slits 8 through which connectors 3 are threaded. The lateral endpoints of the posteriorly-oriented edge of the wound dressing material terminate in tabs 9 that self-adhere with the anteriorly-oriented face of the backing material 4b within the graphical features region 5. A detailed view of distal components of the system is shown in FIG. 1b.

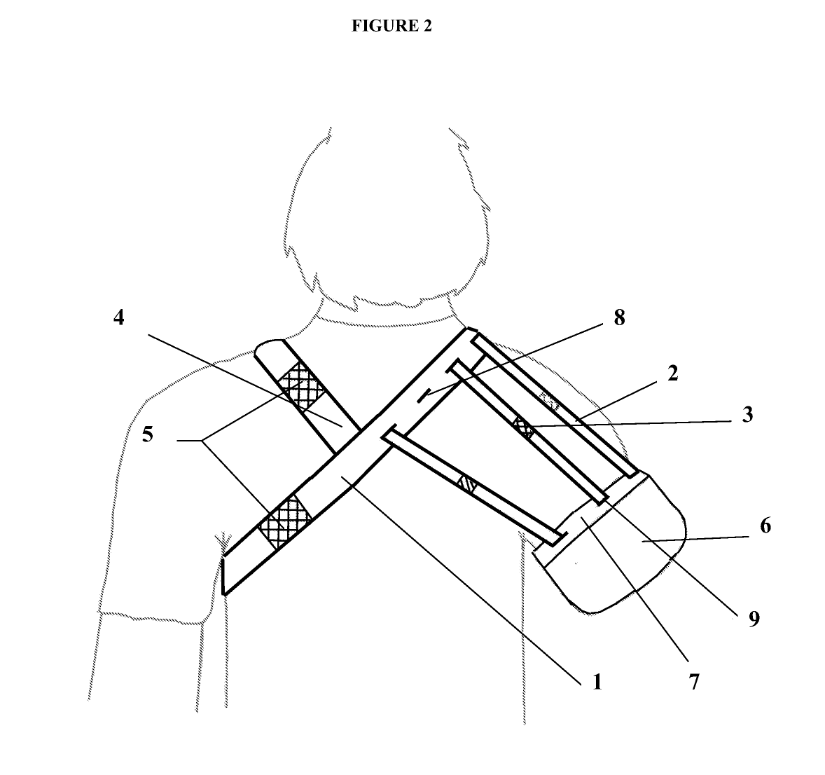

Referring now to FIG. 2, there is generally illustrated an article of manufacture (e.g., a wound dressing system) configured for use with a subject having an upper extremity amputation (e.g., an arm amputation). The embodiment includes a first stabilizing belt 1 which loops across the torso of a subject diagonally from a shoulder area to the armpit region of the opposite side of the torso. The first stabilizing belt intersects with a second stabilizing belt 4, which forms an incomplete “U”-shaped loop over the opposite shoulder relative to the shoulder directly contacted by the first stabilizing belt. Both stabilizing belts 1, 4 comprise hook-and-loop fasteners 5, which confer the ability to adjust the length of each stabilizing belt individually for customization of fit. The first stabilizing belt 1 comprises a plurality of slits 8, through some of which are threaded connectors 2. Additional slits permit a user (healthcare professional, patient) to choose slits for connector threading, further permitting customization of fit. Connectors 2 comprise hook-and-loop fasteners 3, which confer the ability to adjust the length of each connector individually for customization of fit. The distal region of the wound dressing system comprises dressing material with an interior wound-contacting face not visible in FIG. 2 and an exterior backing material 6. The dressing material reinforced edges 7 comprise slits 9 through which connectors are threaded.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the medical arts or related fields are intended to be within the scope of the following claims.

I claim:

1. A dressing system for treatment of a subject with an amputation comprising:
a) a stabilizing belt configured to encompass a portion of said subject's body, wherein said portion of said subject's body is not an amputation stump,
b) dressing material configured to contact an amputation site of said subject, and
c) one or more connectors, said one or more connectors contacting and spanning said stabilizing belt and said dressing material.

2. The dressing system of claim 1, wherein said stabilizing belt is configured to encompass a location selected from the group consisting of torso, waist, hips, neck, and chest.

3. The dressing system of claim 1, wherein said amputation is selected from the group consisting of an upper limb amputation, a lower limb amputation, and a digit amputation.

4. The dressing system of claim 1, further comprising backing material in contact with said dressing material.

5. The dressing system of claim 1, wherein said one or more connectors comprises a fastener selected from the group consisting of hooks, eyes, buttons, hook-and-loop fasteners, adhesive, pins, tabs, snaps, clasps, clamps, carabiners, self-gripping devices, zippers, prongs, eyelets, poppers, and ties.

6. The dressing system of claim 5, wherein said fastener is configured to connect said one or more connectors with said dressing material.

7. The dressing system of claim 4, wherein said backing material further comprises at least one fastener selected from the group consisting of hooks, eyes, buttons, hook-and-loop fasteners, adhesive, pins, tabs, snaps, clasps, clamps, carabiners, self-gripping devices, zippers, prongs, eyelets, poppers, and ties.

8. The dressing system of claim 1, wherein said dressing system comprises graphical elements, wherein said graphical elements serve as visual reference points for adjustment of fit.

9. The dressing system of claim 1, wherein said stabilizing belt is configured to be adjustable in its length.

10. The dressing system of claim 1, wherein at least one of said one or more connectors is configured to be adjustable in its length.

11. A method for dressing a wound, comprising, apply the dressing system of claim 1 to a wound.
